(19) United States
(12) Patent Application Publication
(10) Pub. No.: US 2017/0007470 A1
(43) Pub. Date: Jan. 12, 2017

(54) SANITARY ARTICLE AND METHOD FOR PRODUCING A SANITARY ARTICLE

(71) Applicant: PAUL HARTMANN AG, Heidenheim (DE)

(72) Inventors: Rudolf GROENER, Soehnstetten (DE); Christian KOCH, Bachhagel (DE); Joerg EILERS, Ulm (DE); Nedzad CIFRIC, Herbrechtingen (DE)

(21) Appl. No.: 15/116,395

(22) PCT Filed: Feb. 23, 2015
(86) PCT No.: PCT/EP2015/053718

§ 371 Date: Aug. 3, 2016

(30) Foreign Application Priority Data
Feb. 28, 2014 (DE) 10 2014 203 742.5

(51) Int. Cl. A61F 13/496; A61F 13/58; A61F 13/49

(52) U.S. Cl. CPC A61F 13/496; A61F 13/49017; A61F 13/49011; A61F 13/58; A61F 2013/583

(57) ABSTRACT

The invention relates to a sanitary article, with an absorption body (10) and with leg openings, wherein elongate elasticating means (12) curved in an arc shape are fixed on both sides outside the absorption body (10) in the product transverse direction (16), resulting in elastication of the leg openings (18), wherein a planar material portion (6) facing towards the body comprises a nonwoven (8), characterized in that the nonwoven (8), in the area of the elasticating means (12) on both sides, and for fixing the latter, has, on its side (22) facing away from the body, a first adhesive coating (24) applied in a strip shape over the whole surface and extending in the product longitudinal direction (14), and in that the nonwoven (8), in the area of the elasticating means (12) on both sides, and for fixing the latter, has, on its side (22) facing away from the body on both sides in the product transverse direction (16) outside the first adhesive coating (24), a second adhesive coating (26) applied in a strip shape over the whole surface and extending in the product longitudinal direction (14), and in that the second adhesive coating (26) is shorter, to the front in the product longitudinal direction (14) and to the rear in the product longitudinal direction (14), than the first adhesive coating (24), i.e. ends before the latter, and in that the elongate elasticating means (12) are fixed by the first and second adhesive coatings (24, 26) between the nonwoven (8) and the planar material portion (4).

The invention relates to a sanitary article, such as, in particular, a diaper, a pants-type diaper, an incontinence pad, having a product longitudinal direction and a product transverse direction, having an absorption body and having bilateral marginal regions which delimit leg openings or lea cutouts, wherein, on both sides outside of the absorption body in the product transverse direction and along at least a part of the bilateral marginal regions, elongated elasticating means are provided and fixed between flat material portions of the sanitary article, so that an elastication along at least a part of the leg openings or leg cutouts results, wherein the elasticating means run in an arcuately curved path along at least a part of the leg openings or leg cutouts, and wherein a body-facing flat material portion comprises a nonwoven fabric.

In sanitary articles having elasticating means which are provided along at least a part of the leg openings or leg cutouts and are typically constituted by filiform or band-shaped elasticating means, the object is to fix these elasticating means reliably between flat materials of the sanitary article. If the elasticating means run in an arcuately curved path, this normally proves more difficult than if in a high-speed machine the extent of the elasticating means runs exactly in the machine direction. Arcuately curved or contoured elasticating means along the leg openings can be fixed by an, in particular clocked, adhesive spray application to one flat material or to both flat materials, between which they are received in a sandwich-like manner. The use of adhesive sprays poses problems, however, within the production machine, since adhesive sprayed in the form of a mist contaminates both the product and the machine or machine parts. The use of adhesive spray and in particular spiral bonding by means of spraying methods under the supply of hot air causes air turbulence within the machine, which leads to contaminations, for instance of the deflection rollers in the region of the spray head. The cleaning complexity of the machine is increased and machine downtimes can arise as a consequence of web breaks. In the case of a clocked adhesive spray application, the clocking can give rise to the build-up of an excess pressure in the region of the spray head, which manifests itself in an excessive discharge of adhesive when the spray valve is opened. It is therefore hard in clocked operation to achieve an even adhesive spray application.

A further problem consists in the fact that, when adhesive is applied directly to thermoplastic film or foil materials, the melting point of a thin film or thin foil lies close to the processing temperature of the adhesive. This poses the risk of melting of the plastics foil. Quality defects can arise in the product. Even if a partial melting of the foil does not lead to leakages, then it still leads to regional hardenings of the foil, which can be felt to be unpleasant. When spiral bonding is used, regions without an adhesive coating constantly arise, where the elasticating means consequently remain unfixed and can subsequently slip accidentally out of position.

The object of the present invention is to define a sanitary article and a method for its production, in which the above portrayed problems do not arise or arise to a lesser degree.

This object is achieved according to the invention by a sanitary article of the type stated in the introduction, which is characterized in that the nonwoven fabric, in the region of the bilateral elasticating means and for the fixing thereof, has on its body-remote side a strip-shaped first adhesive coating, which is applied over the whole surface and is extended in the product longitudinal direction, and in that the nonwoven fabric, in the region of the bilateral elasticating means and for the fixing thereof, has on its body-remote side, on both sides outside of the first adhesive coating in the product transverse direction, a strip-shaped second adhesive coating, which is applied over the whole surface and is extended in the product longitudinal direction and which in the product transverse direction partially overlaps with the first adhesive coating or directly adjoins the first adhesive coating or is distanced from the first adhesive coating outwardly in the product transverse direction, and in that the second adhesive coating, to the fore in the product longitudinal direction and to the rear in the product longitudinal direction, respectively shorter than the first adhesive coating, i.e. ends before the latter, and as a consequence of lateral contouring of the bilateral marginal regions for the formation of the leg openings or leg cutouts can also be interrupted or contoured, and in that the elongated elasticating means are fixed by the first and the second adhesive coating between the nonwoven fabric and a, in relation to the nonwoven fabric, body-remote flat material portion (claim 1).

An adhesive spray coating is therefore avoided, and instead a strip-shaped first and second adhesive coating, which has been applied over the whole surface and is extended in the product longitudinal direction, is provided. By an adhesive coating which has been applied in the shape of a strip and over the whole surface is understood an application of molten adhesive material, also referred to as a contact gluing. The liquid adhesive material is in this case applied from a typically slit-shaped adhesive discharge opening, arranged at least close to the nonwoven fabric, of a gluing apparatus, directly through to the nonwoven fabric. It is thus not sprayed via nozzles, but is applied to some extent as a film directly to the nonwoven fabric material, in the shape of a strip and over the whole surface.

The second adhesive coating can overlap with the first adhesive coating, in the product transverse direction, in particular by no more than 10 mm, in particular no more than 7 mm and further particularly no more than 5 mm or can directly adjoin the first adhesive coating, or can be distanced by no more than 10 mm, in particular no more than 7 mm, in particular no more than 5 mm, from the first adhesive coating. Since the second adhesive coating, to the fore and rear in the product longitudinal direction, is respectively shorter than the first adhesive coating, starting from a longitudinal center axis of the sanitary article respectively outside of the first adhesive coating in the product transverse direction, an adhesive-free region is formed to some extent in an imaginary extension of the strip-shaped second adhesive coating. If in this region the elasticating means are guided in the product longitudinal direction during the production of the sanitary article, then they are not fixed in this region with the flat materials. They can then be deadened in terms of their elasticating effect, i.e. deelasticated, by inherently standard techniques, in particular can be cut or can be deelasticated through the use of ultrasound, laser or otherwise.

By contrast, in the region in which the elasticating means run and are extended in the first and the second adhesive coating they are reliably fixed. The elasticating means here extend, in particular with their arcuately curved path, in the first adhesive coating, and make their way from there along their wider extent into the second adhesive coating. From there, they can extend into the aforementioned adhesive-free region, and there follow, in particular linearly, the further product longitudinal direction toward the rear and front.

The elasticating means can thus be guided and introduced between the flat materials such that the arcuately curved path of the bilateral elasticating means runs at least through the first adhesive coating and is fixed (claim 2).

According to a further embodiment of the invention, a linear or straight path of the elasticating means, which adjoins the arcuate path of the elasticating means, runs only through the second adhesive coating and is fixed by the latter (claim 3).

As the elasticating means, filiform or band-shaped elasticating means, such as rubber or polyether polyurethane or polyester polyurethane threads, preferably elastic threads such as Lycra®- or Spandex® threads, are preferably used. The elasticating means preferably have a thickness of 300-1500 dtex, in particular of 500-1200 dtex, further particularly of 500-900 dtex. The elasticating means are preferably fixed under a pretension of 1.5-6.0, in particular of 2.5-4.5, between the nonwoven fabric and the body-remote flat material portion.

It proves advantageous if the first adhesive coating has in the product transverse direction a width of at least 20 mm, in particular of at least 25 mm, and of no more than 40 mm, in particular of no more than 35 mm (claim 4).

In addition, it proves advantageous if the first and/or the second adhesive coating has a weight per unit area of at least 1 g/m², in particular of at least 2 g/m², in particular of no more than 20 g/m², in particular of no more than 10 g/m², in particular of no more than 7 g/m², and further particularly of 2-5 g/m² (claim 5).

In addition, it proves advantageous that the second adhesive coating has in the product transverse direction a width of at least 5 mm, in particular of at least 10 mm, in particular of at least 15 mm and of no more than 30 mm, in particular of no more than 25 mm, and further particularly of no more than 20 mm (claim 6).

It proves particularly advantageous if the second adhesive coating, to the fore in the product longitudinal direction and to the rear in the product longitudinal direction, is respectively shorter by a distance (D) which amounts to at least 15% in particular at least 20% and no more than 35%, in particular no more than 30% of the total length (L₁) of the first adhesive coating in the product longitudinal direction (claim 7). This distance by which the second adhesive coating is respectively shorter at front and rear than the first adhesive coating forms the aforementioned adhesive-free region on both sides outside of the first adhesive coating.

In a refinement of the invention, it proves advantageous if the nonwoven fabric has on its body-remote side, and on both sides outside of the second adhesive coating and outside of the bilateral elasticating means in the product transverse direction, a strip-shaped third adhesive coating applied over the whole surface and extended in the product longitudinal direction (claim 8). In the product transverse direction, the third adhesive coating here partially overlaps with the second adhesive coating or is directly contiguous to the second adhesive coating or is distanced from the second adhesive coating outwardly in the product transverse direction. The third adhesive coating can be overlapped in the product transverse direction, in particular at most up to half the width of the third adhesive coating, by the second adhesive coating, or can directly adjoin the second adhesive coating or be distanced from the second adhesive coating outwardly in the product transverse direction, with a distance of no more than half the width of the third adhesive coating.

The third adhesive coating here has in particular a width of 2-10 mm. In particular, the third adhesive coating has a weight per unit area of at least 1 g/m², in particular of at least 2 g/m², in particular of no more than 20 g/m², in particular of no more than 10 g/m², and further particularly of 4-8 g/m².

In a refinement of this inventive concept, it proves advantageous that in the product transverse direction between the first and the third adhesive coating and at the rear and front in the product longitudinal direction, a channel-shaped adhesive-free portion is formed between the nonwoven fabric and the flat material portion arranged, for this purpose, facing away from the body (claim 9). This adhesive-free portion is that already aforementioned region where the second adhesive coating is not provided, because the second adhesive coating, to the fore and rear in the product longitudinal direction, ends before the first adhesive coating, i.e. is configured shorter than this.

With regard to the third adhesive coating, it proves advantageous if the third adhesive coating, at least in some sections, is configured marginally flush with longitudinal margins of the nonwoven fabric (claim 10). In this way, a marginally flush joining, i.e. bonding of the nonwoven fabric to the, with respect thereto, body-remote flat material can be achieved. As a result, a further barrier to liquid transport in the product transverse direction can be achieved. In this way, capillary liquid transmission in the product transverse direction can be further deterred.

The mentioned bilateral leg openings or leg cutouts can essentially be delimited by linearly, i.e. rectilinearly extended margins, or by arcuately contoured margins. Both are typically realized by lateral contouring of the flat material webs, in particular by the use of cutting rolls. In this respect, it proves advantageous the second adhesive coating, and in particular additionally the first, adhesive coating, through the lateral contouring of the bilateral marginal regions to form leg openings or leg cutouts, are in some sections configured marginally flush with longitudinal margins of the nonwoven fabric (claim 11). This is obtained if the bilateral contour cut for forming the leg openings or leg cutouts runs through the first and, in particular, also the second adhesive coating. A marginally flush bonding of the nonwoven fabric to the flat material arranged, for this purpose, facing away from the body is also hereby achieved, which, in this region too, deters unwanted liquid transport in the product transverse direction.

It can prove advantageous if the first and the second adhesive coating differ from each other by way of different adhesive materials and/or different weight per unit area (claim 12). In this way, the specific requirements can he better met. For instance, the emdedment of arcuately curved elasticating means preferably requires an adhesive with stronger adhesive force and/or higher weight per unit area than is necessary for the fixing of linearly extended elasticating means. It can also be borne in mind that the first and the second adhesive coating have a dual function, they fix the elasticating means and connect the body-facing nonwoven fabric to the, with respect thereto, body-remote flat material.

It further proves advantageous if the nonwoven fabric, on its body-remote side and in the product transverse direction, has as a fourth adhesive coating adhesive tracks applied between the bilateral first adhesive coating over the whole surface and extended in the product longitudinal direction and preferably in parallel (claim 13). Also the adhesive material and/or the weight per unit area of the third or fourth adhesive coating can differ from one another and from those of the first or second adhesive coating. The function of the third and fourth adhesive coating definitely differs from that of the first and the second adhesive coating, insofar as they are not intended to fix the elasticating means, but substantially serve to fix the body-facing nonwoven fabric with the, with respect thereto, body-remote fiat material portion, or to fix components, e.g. of an absorption body, which are present between the body-facing nonwoven fabric and the body-remote flat material portion.

In this context, it proves advantageous if the width of the adhesive tracks of the fourth adhesive coating is 1-5 mm, in particular 2-5 mm, in particular 2-4 mm, and/or that the distance apart of the adhesive tracks of the fourth adhesive coating is 2-20 mm, in particular 2-10 mm, in particular 2-6 mm, in particular 2-4 mm (claim 14).

For the first, second, third and/or fourth adhesive coating, preferably a hotmelt adhesive, further particularly a hydrophobic hotmelt adhesive, is used.

The body-facing flat material portion comprises according to the invention a nonwoven fabric. Further advantageously, the body-facing flat material portion can have in some sections, or in layers, further materials, such as for instance, perforated foils.

It proves particularly advantageous if the nonwoven fabric comprises a spunbonded material, a laminate of spunbonded layers (S) and meltblown layers (M) or a staple fiber nonwoven material, or combinations thereof, and has in particular a weight per unit area of at least 6 g/m², in particular of at least 10 g/m², in particular of no more than 30 g/m², in particular of no more than 20 g/m² (claim 15). Particularly advantageously, the nonwoven fabric forms a body-contacting topsheet layer of the sanitary article.

The, with respect to the nonwoven material, body-remote flat material portion, which, together with the nonwoven fabric and the adhesive, fixes the elasticating means, can advantageously comprise a thermoplastic foil or film layer, in particular a microporous foil or a nonwoven fabric-foil laminate (claim 16).

According to the invention, it proves particularly advantageous if, for the fixing of the elasticating means, only the aforementioned first and second adhesive coating is used, i.e. if in the region of the elasticating means no direct gluing of the body-remote flat material portion, but only of the, with respect thereto, body-facing nonwoven fabric, is realized (claim 17). In this way, the body-remote flat material portion, which is usually formed of a thermoplastic foil or film layer, is then better protected from hot adhesive material, so that the problems, portrayed in the introduction, of the melting of thermoplastic material can be better prevented, it proves particularly advantageous if at no place is molten adhesive material applied from an applicator directly to the thermoplastic flat material portion, but only after application to other flat materials, such as, for example, the body-facing nonwoven fabric. With regard to the problems of contamination within the machine, it also proves advantageous if no spray gluing of the elasticating means is performed, especially as this, as a consequence of the areal contact gluing in the form of the first and the second adhesive coating, is also no longer necessary.

In addition, it can prove advantageous if the first and/or the third and/or the fourth adhesive coating extend in the product longitudinal direction up to longitudinal ends of the sanitary article (claim 18). In this case, the particular adhesive coatings can be endlessly applied in the machine direction, which always proves advantageous in production engineering terms. In this case, the nonwoven fabric contained in the body-facing flat material portion, and the, in relation thereto, body-remote flat material, at the same time form the shell or chassis materials of the sanitary article, which define and delimit the sanitary article in the flatly spread, i.e. wide spread, state (apart from, for example, protruding closure tabs).

Another embodiment is characterized by a layered structure comprising the absorption body and forming the bilateral marginal regions, in which the elongated elasticating means are provided along at least a part of the leg openings or leg cutouts (claim 19). This layered structure comprising the absorption body can here be connected to further chassis-forming components of the sanitary article, or supplemented by these in the aforesaid planar projection. In this sense, it would be conceivable that merely the layered structure comprising the absorption body is configured in the previously stated manner with a first and second adhesive coating and, if need be, third and fourth adhesive coating, and is separately produced endlessly. In such a case, these layered structures can be individually obtained by separation transversely to the direction of production and are fed to further components in a high-speed production machine. In an extrapolation of this inventive concept, protection is claimed for a sanitary article having a layered structure which comprises an absorption body and to which lateral portions are fitted, in particular lateral portions having closure tabs for provision as an operable and/or closable sanitary article. In another extrapolation of this inventive concept, protection is claimed for a sanitary article having a front abdominal portion and a rear spinal portion, which are formed by separate components that are spaced apart in the product longitudinal direction, yet, for the formation of an abdominal and spinal band passing in the transverse direction or hip circumferential direction, are factory-connected to each other at bilateral side seam regions with a hip opening that is dosed in the hip circumferential direction, and having a crotch portion, which comprises the absorption body and extends in the product longitudinal direction between the abdominal portion and the spinal portion and bridges these and is non-detachably joined to the abdominal portion and to the spinal portion in a respective overlap region, wherein the abdominal portion, the spinal portion and the crotch portion jointly delimit the leg openings, wherein in the abdominal portion and in the spinal portion are provided further elasticating means, which run in the product transverse direction and areally elasticate the abdominal portion and the spinal portion, and wherein the crotch portion forms the layered structure comprising the absorption body (claim 20). This sanitary article is thus formed of three components, namely the front abdominal portion, the rear spinal portion and the crotch portion, wherein the latter is configured as a separately produced unit according to, the invention, i.e. with a first and second adhesive coating in the previously described manner, and in particular and advantageously also has the third and/or fourth adhesive coating.

The crotch portion, i.e. the layered structure, is here fixed in a respective overlap region with the abdominal portion and the spinal portion, in particular by means of adhesive. The manner, arrangement and configuration of the adhesive fixing is described in DE 102007055524 A1, and the relevant content of this DE 102007055524 A1 is incorporated by reference into the present Application. This manner, arrangement and configuration of the adhesive fixing is preferred, but not essential. Further preferably, the arrangement and configuration of the adhesive fixing can be realized as described in DE 102012208393 A1, and the relevant content of this DE 102012208393 A1 is incorporated by reference into the present Application.

It can also prove advantageous if the further elasticating means in the abdominal portion and the spinal portion extend at a distance apart and parallel to each other in the product transverse direction or hip circumferential direction of the sanitary article and thus areally elasticate the abdominal portion and the spinal portion. In addition, it can prove advantageous if, in a crotch-side region, facing the leg openings, of the abdominal portion and of the spinal portion, are provided elasticating means, which, in particular starting from the two side seam regions, extend in the direction of a longitudinal center axis of the incontinence article and hereupon extend into the overlap region of crotch portion and abdominal portion or of crotch portion and spinal portion, wherein they can there in particular, be stripped of their elasticating effect, in particular can be cut.

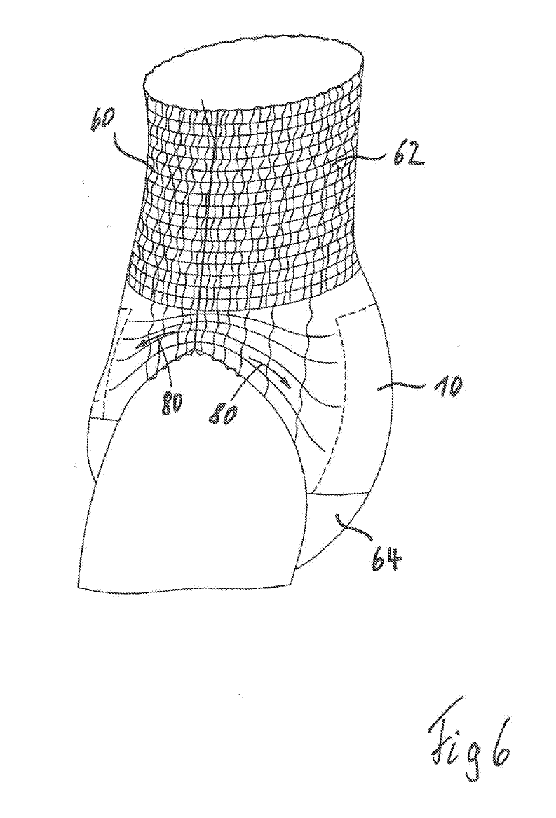

The configuration and arrangement of the elasticating means running in the hip circumferential direction and of the elasticating means provided in particular in a crotch-side region, facing the leg openings, of the abdominal portion and spinal portion are described in DE 102007055524 A1, so that the relevant content of DE 102007055524 A1 is incorporated by reference into the present Application. This also applies to the stress distribution of the elasticating means provided in a crotch-side region, facing the leg openings, of the abdominal portion and spinal portion, in particular as a result of decreasing surface restoring forces in the regions, facing the leg openings, of the abdominal portion or spinal portion (such as is indicated in FIG. 6 in the direction of the arrows 80).

It further proves advantageous if the first and/or the third and/or the fourth adhesive coating extends in the product longitudinal direction up to longitudinal ends of the layered structure comprising the absorption body, i.e. up to longitudinal ends of the crotch portion (claim 21).

The subject of the invention is also a method for producing a sanitary article or a layered structure, comprising an absorption body, of a sanitary article or, more particularly, a method for fixing elongated elasticating means in, the course of production of a sanitary article having the features of claim 22. A method having the following features is thus proposed:
feeding of an endless nonwoven fabric, forming the body-facing flat material portion, in a machine direction which corresponds to the later product longitudinal direction,
continuous all-over application of the first adhesive coating, in the machine direction, to the body-remote side of the nonwoven fabric in the bilateral marginal regions.
clocked all-over application of the second adhesive coating, in the machine direction, to the body-remote side of the nonwoven fabric in the bilateral marginal regions outside of the first adhesive coatings in the product transverse direction, such that the second adhesive coating, in the product transverse direction, partially overlaps with the first adhesive coating or directly adjoins the first adhesive coating, or is distanced from the first adhesive coating in the product transverse direction, wherein the second adhesive coating is applied in a clocked manner such that, to the front and rear in the product longitudinal direction, it is shorter than the first adhesive coating, i.e. ends before the latter,
feeding of en endless at material web, forming the body-remote flat material portion, in the machine direction,
feeding of the bilateral elasticating means in the machine direction between the nonwoven fabric and the body-remote flat material web, at least in some sections, in the region of the first and the second, adhesive coating,
bringing together of the endless nonwoven fabric and the body-remote flat material web, with the interposition of the elasticating means, and fixing of elasticating means by the first and the second adhesive coating,
deelastication of the elasticating means in a region which has remained free of adhesive in the course of the clocking of the second adhesive coating in the product longitudinal direction.

The aforesaid nonwoven fabric is fed in the form of an, in the machine direction, endless nonwoven fabric web in accordance with the flat material web forming the body-remote flat material portion. The respective all-over adhesive coating is once again applied, as described in the introduction, as a contact gluing. The advantages obtained by the method according to the invention have respectively already been discussed in the introduction in connection with the sanitary article according to the invention, so that reference is made thereto. It should further be mentioned that for the deelastication of the elasticating means, in an advantageous process a respective elasticating means, for instance, needs to be parted only at a single place, so that it can contract freely over the length of the region which has remained free of adhesive and in this way loses its elasticating effect on the chassis materials, since in said region it is not, of course, adhesively connected to these. In this context, it proves advantageous if this region is delimited in the product transverse direction on the one hand by the first adhesive coating, and outwardly by the third adhesive coating. In this way, a type of channel is created for the elasticating means, which channel is extended in the product longitudinal direction.

The method is advantageously further characterized by all-over application of a third adhesive coating, in the machine direction, to the body-remote side of the nonwoven fabric in the bilateral marginal regions outside of the second adhesive coating and outside of the bilateral elasticating means in the product transverse direction (claim 23). The application of the third adhesive coating is here such that the third adhesive coating, in the product transverse direction, here partially overlaps with the second adhesive coating overlaps or is directly contiguous to the second adhesive coating, or is distanced from the second adhesive coating outwardly in the product transverse direction. In the application of the third adhesive coating, the third adhesive coating is overlapped in the product transverse direction, in particular at most up to half the width of the third adhesive coating, by the second adhesive coating, or directly adjoins the second adhesive coating, or is distanced from the second adhesive coating outwardly in the product transverse direction by a distance of no more than half the width of the third adhesive coating.

The method is advantageously further characterized by all-over application of a fourth adhesive coating in the machine direction to the body-remote side of the nonwoven fabric onto a region in the product transverse direction between the bilateral first adhesive coating in the form of adjacently extended adhesive tracks, in particular having a width of 1-5 mm, in particular 2-5 mm, in particular 2-4 mm, and at a distance apart of the adhesive tracks of 2-20 mm, in particular 2-10 mm, in particular 2-6 mm, in particular 2-4 mm (claim 24).

It here proves advantageous if the third adhesive coating and/or the fourth adhesive coating are applied continuously in the machine direction (claim 25).

It proves advantageous if, for the fixing of the elasticating means, only the first and a second adhesive coating are used and the body-remote flat material we remains uncoated in this region (claim 26).

It is further proposed that, for the application of the first and the second adhesive coating, in particular also for the application of the third and/or fourth adhesive coating, an adhesive coating apparatus, which in closest contact to the nonwoven fabric delivers adhesive material from, in particular, slit-shaped adhesive discharge openings, is used (claim 27). In this context, it proves advantageous if the adhesive coating apparatus comprises a delivery pressure controlling and preferably delivery pressure regulating device. In this way, a clocked adhesive coating can be realized without the need to use further mechanical actuating components which, for instance, must raise an adhesive coating head. The adhesive coating apparatus can be provided for the individual or parallel application of the first, second, third and/or fourth adhesive coating. Preferably, the adhesive coating apparatus enables the simultaneous and parallel application at least of the first and third and/or fourth adhesive coating.

For the configuration of the leg openings or leg cutouts, it proves advantageous if, after the nonwoven fabric and the body-remote flat material web have been brought together, contour cuts, conducted in the machine direction, are made on, both sides in order to form the leg openings or the leg cutouts (claim 28).

It further proves advantageous if, transversely to the machine direction, a separation step for the formation of individual sanitary articles is performed. In particular, it proves advantageous if the deelastication of the elasticating means is performed together in a separation step, realized transversely to the machine direction for the formation of individual sanitary articles.

In addition, it proves advantageous if, with the method according to the invention, an endless web of layered structures, respectively comprising an absorption body, is produced, which endless web comprises the body-remote flat material web and the body-facing nonwoven fabric and an endless succession of absorption bodies, fed one after another in the machine direction, which are conveyed and arranged between the body-remote flat material web and the body-facing nonwoven fabric, and that, transversely to the machine direction, a separation cut for the formation of the layered structures is performed, and that lateral portions, in particular lateral portions having closure tabs for provision as operable and/or closable sanitary articles, are fitted to the separated layered structures.

In addition, it proves advantageous if, with the method according to the invention, an endless web of layered structures, respectively comprising an absorption body, is produced, which endless web comprises the body-remote flat material web and the body-facing nonwoven fabric and an endless succession of absorption bodies, fed one after another in the machine direction, which are conveyed and arranged between the body-remote flat material web and the body-facing nonwoven fabric, and that, transversely to the machine direction, a separation cut for the formation, of the layered structures is performed, that the separated layered structures are rotated through 90° and are brought into bridging arrangement with an abdominal portion web and a spinal portion web and are non-detachably fixed in the overlap region with the abdominal portion web and the spinal portion web (claim 29).

Further details, features and advantages of the invention emerge from the appended patent claims and from the graphic representation and the following description of the method according to the invention and of sanitary articles according to the invention.

In the drawing:

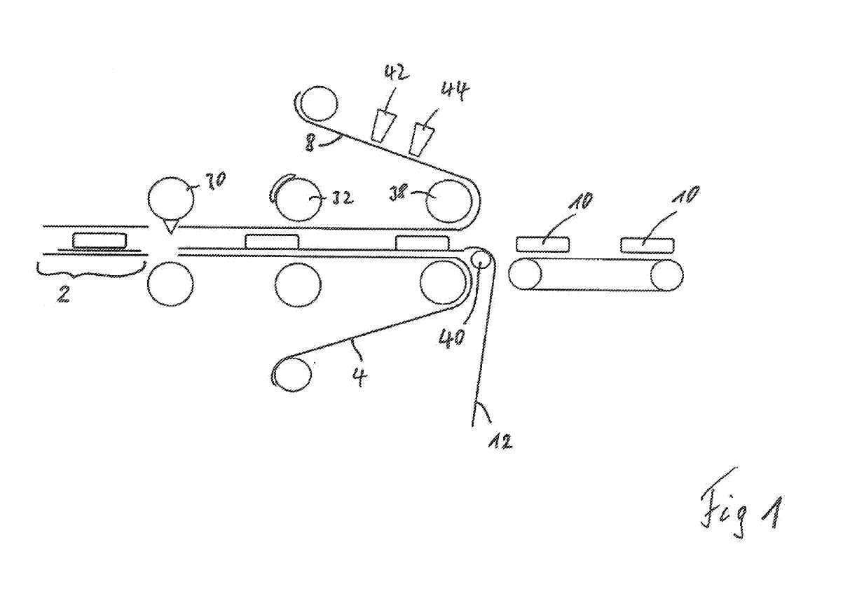

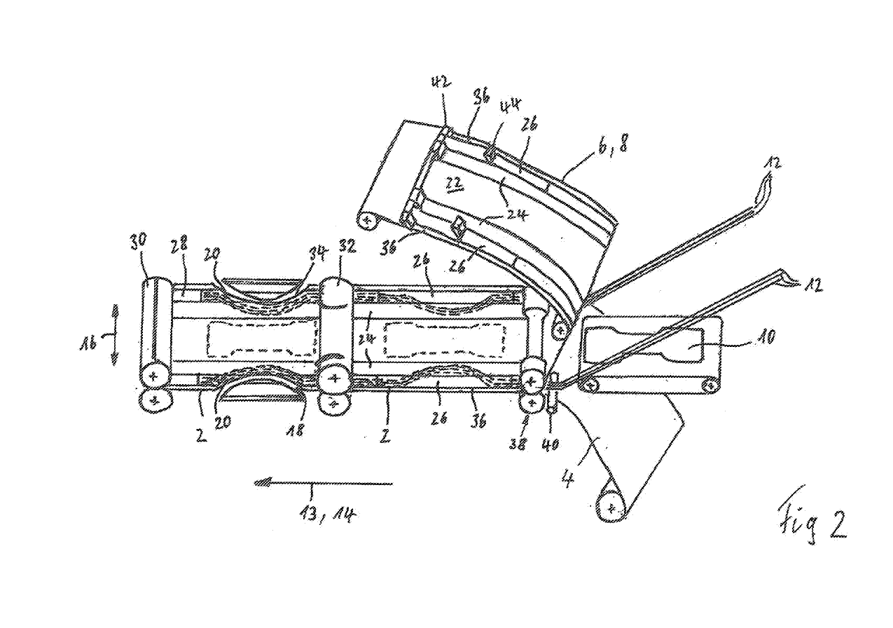

FIGS. 1 and 2 show schematic view or illustration the production method according to the invention;

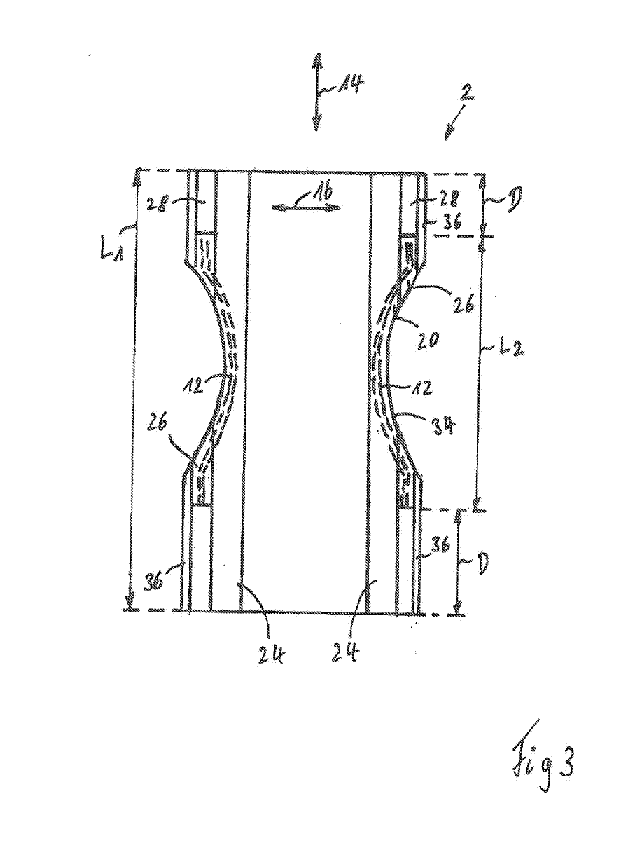

FIG. 3 shows a top view of layered structure according to the invention (schematically for representation of the strip-shaped adhesive coating);

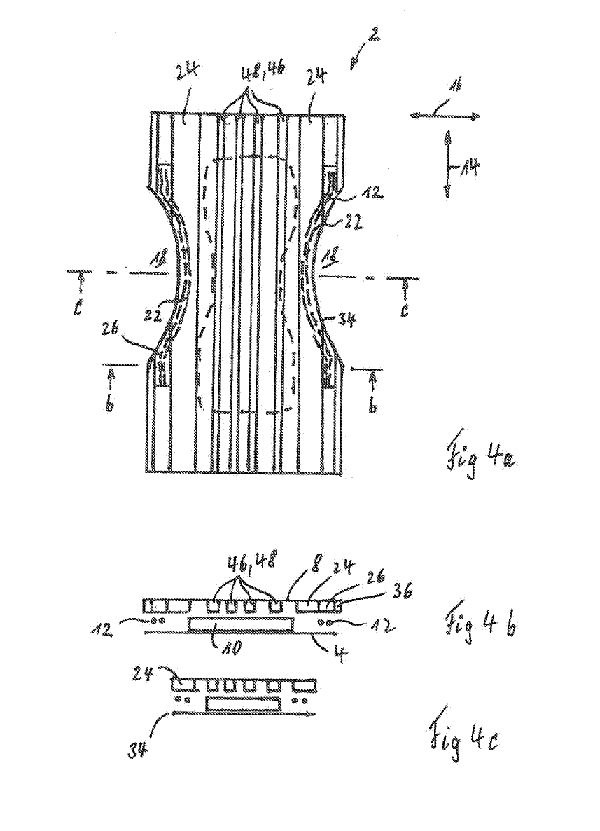

FIGS. 4a-c show a top view of a further embodiment of a layered structure according to the invention with two sectional views (schematically for representation of the strip-shaped adhesive coating);

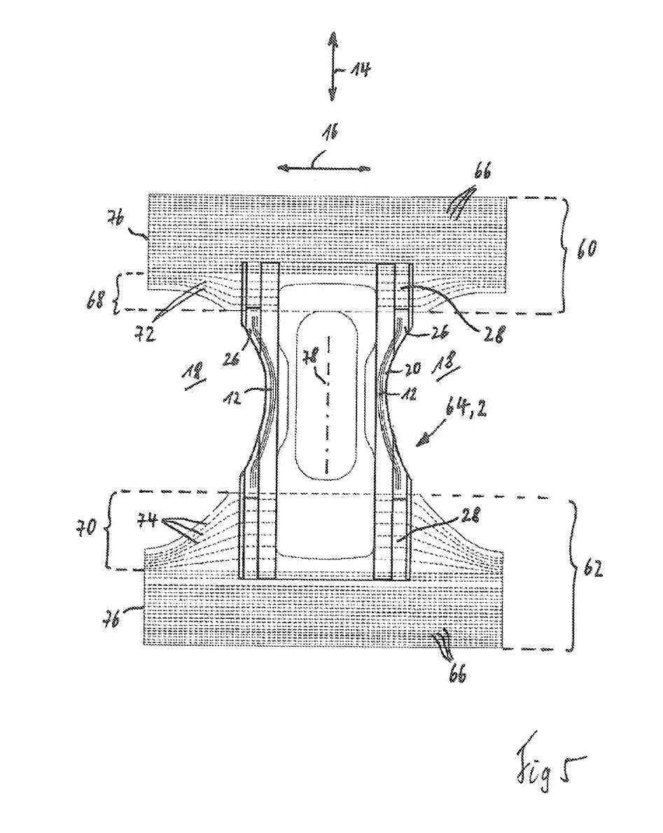

FIG. 5 shows a top view of sanitary article according to the invention, having a crotch portion in accordance with the layered structure according to FIG. 3;

FIG. 6 shows a perspective view of the sanitary article according to FIG. 5;

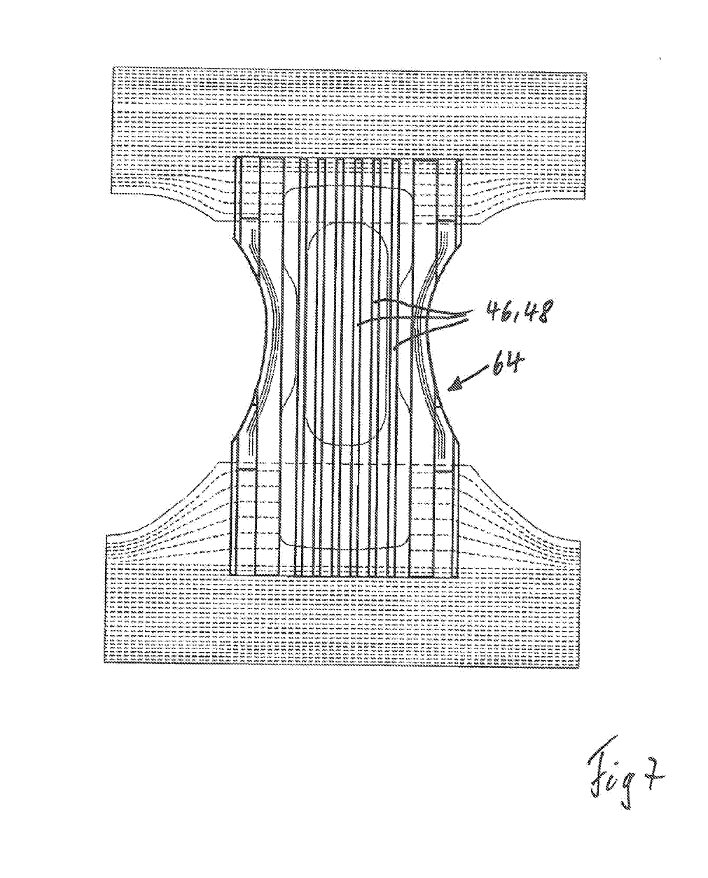

FIG. 7 shows a top view of a further sanitary article according to the invention, having a crotch portion in accordance with the layered structure according to FIG. 4.

FIGS. 1 and 2 illustrate the method according to the invention for producing layered structures 2, comprising an absorption body, in endless succession, which layered structures are represented in separated form in FIGS. 3 and 4. For instance, these layered structures 2 can also be constituted by sanitary articles which are inherently ready for use, for instance in the form of incontinence pads or incontinence liners or—following fitting of lateral closure tabs or lateral portions with closure tabs—by openable and/or closable diapers. The layered structures produced as represented in FIGS. 1 and 2 comprise at least the following components: a body-remote flat material portion 4, in particular in the form of a liquid-impermeable thermoplastic foil material, a body-facing flat material portion 6 in the form of a nonwoven fabric 8, an absorption body 10 conveyed between the flat material portion 4 and the nonwoven fabric 8, and elasticating means 12, extended on both sides of the absorption body, which, like the absorption body 10, are accommodated in a sandwich-like manner between the flat material portion 4 and the nonwoven fabric 8 and are fixed by an adhesive coating (yet to be described). Reference symbol 13 denotes a machine direction or feed direction, which is aligned with, a product longitudinal direction 14 and is orthogonal to a product transverse direction 16. Although the elasticating means 12, during the production process, extend in the machine direction 13, they are not continuously linear, but extend in an arcuately curved path along at least a part of the leg openings or leg cutouts 18.

For the fixing of the elasticating means 12 in marginal regions 20 between the flat material portion 4 and the, with respect thereto, body-facing nonwoven fabric 8, areally applied adhesive is used. For this, a strip-shaped first adhesive coating 24, which is extended in the product longitudinal direction 14, is applied to a body-remote side 22 of the nonwoven fabric 8 is applied over the whole surface. The first adhesive coating 24, which is applied on both sides of the absorption body 10 in the region of the intended arrangement of the elasticating means 12, is applied in the machine direction 13 preferably endlessly, i.e. in continuous passage, to the nonwoven fabric 8.

Outside of the first adhesive coating 24 in the product transverse direction 16, a second adhesive coating 26 is applied, though not in continuous passage in the machine direction 13, but in a clocked, i.e. interrupted, manner. This second adhesive coating 26 is applied in a clocked manner to the nonwoven fabric 8 such that, both to the front and rear in the product longitudinal direction 14, it ends before the first adhesive coating 24, i.e. is shorter than the latter in the product longitudinal direction 14. It can best be seen from FIG. 3 that the second adhesive coating 26, at the front and rear respectively in the product longitudinal direction 14, is shorter by a distance D than the first adhesive coating 24. The total length thereof is denoted by L1. The total length of the second adhesive coating 26 is marked in FIG. 3 with L2. Advantageously, the distance D at front and rear respectively amounts to between 15% and 30% of the total length L1 of the first adhesive coating 24. In this way, in the layered structure 2, an adhesive-free region 28 of length D is formed between the body-remote flat material portion 4 and the nonwoven fabric 8 in the product longitudinal direction 14. If, as represented in FIG. 2, the elasticating means 12 are guided through these adhesive-free regions 28 in the machine direction 13 or the product longitudinal direction 14, then they are there not fixed with the chassis-forming shell materials, i.e. the flat material portion 4 and the nonwoven fabric 8. This proves advantageous, since the elasticating means 12 in this adhesive-free region 28 can be deelasticated relatively easily by process engineering means, by being cut at just one place or being otherwise stripped of their elasticating effect. Since the elasticating means 12 have been conveyed in the pretensioned state between the shell materials and have been fixed in a laminate-like manner in the glue bed of the first and the second adhesive coating 24, 26, they can contract, when cut, into the configuration represented in FIGS. 2 to 4. A cutting station for cutting through the elasticating means 12, and preferably at the same time separating the layered structures 2, is denoted in FIG. 2 by the reference symbol 30. A cutting station for contouring the leg openings or leg cutouts 18 bears the reference symbol 32. It can be seen from FIG. 2 that, by means of the cutting station 32, a contour cut is conducted through the first and the second adhesive coating 24, 26, so that, in the case represented by way of example, the first and the second adhesive coating 24, 26 are configured marginally flush with longitudinal margins 34 of the nonwoven fabric 8 in the region of the contour cut.

In the preferred embodiment of the invention which is represented by way of example, a strip-shaped third adhesive coating 36, which is likewise applied over the whole surface and is extended in the product longitudinal direction 14, is provided onto the body remote side 22 of the nonwoven fabric 8 on both sides outside of the second adhesive coating 26 and outside of the bilateral elasticating means 12 in the product transverse direction 16. The third adhesive coating 36 preferably runs marginally flush with the straight longitudinal margins 34 of the nonwoven fabric prior to the making of the contour cuts for the formation of the leg openings or leg cutouts 18. The flat material portion 4 and the nonwoven fabric 8 are hereby connected to each other continuously, i.e. without interruption, in the product longitudinal direction 14. The third adhesive coating 36 fulfills, however, a further function. It delimits the aforementioned adhesive-free region 28. Thus the respective adhesive-free region 28 is delimited from the inside in the product transverse direction 16 by the respective first adhesive coating 24, and from the outside in the product transverse direction 16 by the third adhesive coating 36. In this way is formed an elongated channel-shaped portion, in which the elasticating means 12 are guided unfixed, and initially in continuous passage, in the machine direction 13. They can therefore be raised and cut in a manner which is known per se, or be cut in the course of a separation cut without the liquid-impermeable flat material portion 4 being hereby affected.

By reference symbol 38 is denoted a nip roll pair the region of the first to third adhesive coating 24, 26, 36 or in the region of the elasticating means 12. By reference symbol 40 are indicated deflection pins, by means of which the elasticating means 12 can be guided back and forth for the formation of the arcuately curved path in the product transverse direction 16. By reference symbol 42 is denoted in FIG. 2 an adhesive coating apparatus for the first adhesive coating 24 and, in the case represented by way of example, also for the third adhesive coating 36. For the second adhesive coating 26, a further adhesive coating apparatus 44 is represented. The adhesive coating apparatuses are here constituted by contact gluing apparatuses, which have a slit-shaped adhesive discharge opening extended in the product transverse direction 16. The adhesive coating apparatuses 42 and 44 can be configured as described in the introduction.

FIG. 4a shows a layered structure 2 corresponding to FIG. 3, wherein a fourth adhesive coating 46, to be precise in the form of a plurality of adhesive tracks 48 extended, in the case represented by way of example, in parallel and in the product longitudinal direction 14, is additionally provided between the, left and the right first adhesive coating 24 in the product transverse direction 16. These adhesive tracks 48 are once again applied in the shape of a strip and over the whole surface, but they advantageously and preferably have a smaller width than the first adhesive coating 24. They serve to fix the nonwoven fabric 8 with an underlying layer, i.e. in particular with the absorption body 10 and with the body-remote flat material portion 4 outside of the absorption body 10 FIGS. 4b and 4c show strongly schematic, non-true-to-scale sectional views, with sectional plane b-b and c-c, respectively, in FIG. 4a, which are intended to illustrate the respective adhesive coating.

Finally, FIG. 5 shows a top view of a further sanitary article according to the invention in pants shape in the flatly spread state, i.e. prior to the connection of lateral longitudinal marginal regions. The pants-shaped sanitary article represented in FIGS. 5 and 6 is made up of three components, namely a front abdominal portion 60, a rear spinal portion 62 and a crotch portion 64 bridging these two in the product longitudinal direction 14 In the case represented by way of example, the crotch portion 64 is constituted by the layered structure 2 described in the preceding figures. The crotch portion 64, i.e. the layered structure 2, is fixed in the respective overlap region with the abdominal portion 60 and the spinal portion 62 by means of adhesive. The manner, arrangement and configuration of the adhesive fixing is described in DE 102007 055524 A1, and the relevant content of this DE 102007055524 A1 is incorporated by reference into the present Application. This manner, arrangement and configuration of the adhesive fixing is preferred, but not essential. Further preferably, the arrangement and configuration of the adhesive fixing can be realized as described in DE 102012208393 A1, and the relevant content of this DE 102012208393 A1 is incorporated by reference into the present Application.

It can be seen in FIGS. 5 and 6 that in the abdominal portion 60 and in the spinal portion 62 are respectively provided elasticating means 66, running in the product transverse direction 16 or in the hip circumferential direction and in parallel, which elasticate the abdominal portion 60 or the spinal portion 62 areally in the product transverse direction 16. In a region 68, 70, facing the leg openings 18, of the abdominal portion 60 and spinal portion 62 respectively are provided elasticating means 72, 74, which, starting from lateral longitudinal seam regions 76, widen in a fan-like manner in the direction of a longitudinal center axis 78. The configuration and arrangement of the elasticating means 66 running in the hip circumferential direction and of the arcuately fanning elasticating means 72, 74 are described in DE 102007055524 A1, so that the relevant content of DE 102007055524 A1 is incorporated by reference into the present Application. This also applies to the stress distribution in the elasticating means 72 and 74 and to the, in the direction of the arrows 80 (FIG. 6), decreasing surface restoring forces in those regions 68, 70 of the abdominal portion 60 and spinal portion 62, respectively, which are facing the leg openings 18.

Finally, FIG. 7 shows a pants-shaped sanitary article in accordance with FIGS. 5 and 6, wherein the crotch portion 64 is configured in accordance with the layered structure according to FIGS. 4a to c, i.e. additionally has a fourth adhesive coating 46 having adhesive tracks 48 extended in the product longitudinal direction 14. Further differences from the sanitary article according to FIG. 6 are non-existent.

In the case of the pants-shaped sanitary article according to FIGS. 5 to 7, it can be seen that the respective second adhesive coating 26, in the product longitudinal direction 14, ends approximately at the start of the overlap of crotch portion 64 and abdominal portion 60 or of crotch portion 64 and spinal portion 62. This means that the elasticating means 12 assigned to the leg openings 18 produce, approximately up to this region, an elastication of the marginal regions 20 delimiting the leg openings 18. It further proves advantageous that the elasticating means 12, though they are fed endlessly in the machine direction 13 and are laminated, do not elasticate the adhesive-free region 28 of the layered structure 2, as a consequence of the cutting operation, in the sanitary article according to FIGS. 5 to 7, this adhesive-free region 28 is arranged in overlap with the abdominal portion 60 or spinal portion 62. In this way, the elasticating means 12, though they are fed endlessly prior to the cutting operation, have no effect which causes disturbance and interferes with the elasticating means 66, 72 and 74 of the abdominal portion 66 and spinal portion 64.

1. A sanitary article, having a product longitudinal direction (14) and a product transverse direction (16), having an absorption body (10) and having bilateral marginal regions (20) which delimit leg openings or leg cutouts (18), wherein, on both sides outside of the absorption body (10) in the product transverse direction (16), and along at least a part of the bilateral marginal regions (20), elongated elasticating means (12) are provided and fixed between fiat material portions (4, 6) of the sanitary article, so that an elastication along at least a part of the leg openings or leg cutouts (18) results, wherein the elasticating means (12) run in an arcuately curved path along at least a part of the leg openings or leg cutouts (18), and wherein a body-facing flat material portion (6) comprises a nonwoven fabric (8), characterized in that the nonwoven fabric (8), in the region of the bilateral elasticating means (12) and for the fixing thereof, has on its body-remote side (22) a strip-shaped first adhesive coating (24), which is applied over the whole surface and is extended in the product longitudinal direction (14), and in that the nonwoven fabric, (8), in the region of the bilateral elasticating means (12) and for the fixing thereof, has on its body-remote side (22), on both sides outside of the first adhesive coating (24) in the product transverse direction (16), a strip-shaped second adhesive coating (26), which is applied over the whole surface and is extended in the product longitudinal direction (14) and which in the product transverse direction (16) partially overlaps with the first adhesive coating (24) or directly adjoins the first adhesive coating, (24) or is outwardly distanced from the first adhesive coating (24) in the product transverse direction (16), and in that the second adhesive coating (26), to the fore in the product longitudinal direction (14) and to the rear in the product longitudinal direction (14), is respectively shorter than the first adhesive coating (24), and as a consequence of lateral contouring of the bilateral marginal regions (20) for the formation of the leg openings or leg cutouts (18) can also be interrupted or contoured, and in that the elongated elasticating means (12) are fixed by the first and the second adhesive coating (24, 26) between the nonwoven fabric (8) and, in relation to the nonwoven fabric (8), body-remote flat material portion (4).

2. The sanitary article as claimed in claim 1, characterized in that the it arcuately curved path of the bilateral elasticating means (12) runs at least through the first adhesive coating (24) and is fixed.

3. The sanitary article as claimed in claim 1, characterized in that a linear path of the elasticating means, which adjoins the arcuate path of the elasticating means (12), runs only through the second adhesive coating and is fixed.

4. The sanitary article as claimed in claim 1, characterized in that the first adhesive coating (24) has in the product transverse direction (16) a width of at least 20 mm.

5. The sanitary article as claimed in claim 1, characterized in that the first and/or the second adhesive coating (24, 26) has a weight per unit area of at least 1 g/m².

6. The sanitary article as claimed in claim 1, characterized in that the second adhesive coating (26) has in the product transverse direction (16) a width of at least 5 mm and of no more than 30 mm.

7. The sanitary article as claimed in claim 1, characterized in that the second adhesive coating (26), to the fore in the product longitudinal direction (14) and to the rear in the product longitudinal direction (14), is respectively shorter by a distance (D) which amounts to at least 15% and no more than 35% of the total length (L₁) of the first adhesive coating (24) in the product longitudinal direction (14).

8. The sanitary article as claimed in claim 1, characterized in that the nonwoven fabric (8) has on its body-remote side (22), and on both sides outside of the second adhesive coating (26) and outside of the bilateral elasticating means (12) in the product transverse direction (16), a strip-shaped third adhesive coating (36) applied over the whole surface and extended in the product longitudinal direction (14).

9. The sanitary article as claimed in claim 8 characterized in that, in the product transverse direction (16) between the first and the third adhesive coating (24, 36) and at the rear and front in the product longitudinal direction (14), a channel-shaped adhesive-free portion (28) is farmed between the nonwoven fabric (8) and the flat material portion (4) arranged, for this purpose, facing away from the body.

10. The sanitary article as claimed in claim 8, characterized in that the third adhesive coating (36), at least in some sections, is configured marginally flush with longitudinal margins (34) of the nonwoven fabric (8).

11. The sanitary article as claimed in claim 1, characterized in that the second adhesive coating (26), through the lateral contouring of the bilateral marginal regions (20) to form leg openings or leg cutouts (18), is in some sections configured marginally flush with longitudinal margins (34) of the nonwoven fabric (8).

12. The sanitary article as claimed in claim 1, characterized in that the first and the second adhesive coating (24, 26) differ from each other by way of different adhesive materials and/or different weight per unit area.

13. The sanitary article as claimed in claim 1, characterized in that the nonwoven fabric (8), on its body-remote side (22) and in the product transverse direction (16), has as a fourth adhesive coating (46) adhesive tracks (48) applied between the bilateral first adhesive coating (24) over the whole surface and extended in the product longitudinal direction (14) and preferably in parallel.

14. The sanitary article as claimed in claim 13, characterized in that the width of the adhesive tracks (48) of the fourth adhesive coating (46) is 1-5 mm, and/or in that the distance apart of the adhesive tracks of the fourth adhesive coating is 2-20 mm.

15. The sanitary article as claimed in claim 1, characterized in that the nonwoven fabric (8) comprises a spunbonded material, a laminate of spunbonded layers (S) and meltblown layers (M) or a staple fiber nonwoven material, or combinations thereof, and has in particular a weight per unit area of at least 6 g/m².

16. The sanitary article as claimed in claim 1, characterized in that the body-remote flat material portion (4) comprises a thermoplastic foil or film layer, an in particular microporous foil, or a nonwoven fabric-foil laminate.

17. The sanitary article as claimed in claim 1, characterized in that, for the fixing of the elasticating means (12), only the first and the second adhesive coating (24, 26) is used.

18. The sanitary article as claimed in claim 1, characterized in that the first and/or a third and/or a fourth adhesive coating (24, 36, 46) extend in the product longitudinal direction (14) up to longitudinal ends of the sanitary article.

19. The sanitary article as claimed, in claim 1, characterized by a layered structure (2) comprising the absorption body (10) and forming the bilateral marginal regions (20), in which the elongated elasticating means (12) are provided along at least a part of the leg openings or leg cutouts (18).

20. The sanitary article as claimed in claim 19, having a front abdominal portion (60) and a rear spinal portion (62), which are formed by separate components that arc spared apart in the product longitudinal direction (14), yet, for the formation of an abdominal and spinal band passing in the transverse direction or hip circumferential direction, are factory-connected to each other at bilateral side seam regions (76) with a hip opening that is closed in the hip circumferential direction, and having a crotch portion (64), which comprises the absorption body (10) and extends in the product longitudinal direction (14) between the abdominal portion (60) and the spinal portion (62) and bridges these and is non-detachably joined to the abdominal portion (60) and to the spinal portion (62) in a respective overlap region, wherein the abdominal portion (60), the spinal portion (62) and the crotch portion (64) jointly delimit the leg openings (18), wherein in the abdominal portion (60) and in the spinal portion (62) are provided further elasticating means (66), which run in the product transverse direction (16) and areally elasticate the abdominal portion (60) and the spinal portion (62), and wherein the crotch portion (64) forms the layered structure (2) comprising the absorption body (10).

21. The sanitary article as claimed in claim 20, characterized in that the first and/or a third and/or the a fourth adhesive coating (24, 36, 46) extend in the product longitudinal direction (14) up to longitudinal ends of the layered structure (2) comprising the absorption body (10).

22. A method for producing a sanitary article as claimed in claim 1, wherein the elongated elasticating means (12), along at least a part of the marginal regions (20) delimiting the bilateral leg openings or leg cutouts (18), are fixed between flat material portions (4, 6) of the sanitary article, comprising the following features:
feeding of an endless nonwoven fabric (8), forming the body facing flat material portion (6), in a machine direction (13) which corresponds to the later product longitudinal direction (14),
continuous all-over application of the first adhesive coating (24), in the machine direction (13), to the body-remote side (22) of the nonwoven fabric (8) in the bilateral marginal regions (20),
clocked all-over application of the second adhesive coating (26), in the machine direction (13), to the body-remote side of the nonwoven fabric (8) in the bilateral marginal regions (20) outside of the first adhesive coating (24) in the product transverse direction (16), such that the second adhesive coating (26), in the product transverse direction (16), partially overlaps with the first adhesive coating (24) or directly adjoins the first adhesive coating (24), or is distanced from the first adhesive coating (24) in the product transverse direction (16), wherein the second adhesive coating (26) is applied in a clocked manner such that, to the front and rear in the product longitudinal direction (14), it is shorter than the first adhesive coating (24),
feeding of an endless flat material web, forming the body-remote flat material portion (4), in the machine direction (13),
feeding of the bilateral elasticating means (2) in the machine direction (13) between the nonwoven fabric (8) and the body-remote fiat material web, at least in some sections, in the region the first and the second adhesive coating (24, 26),
bringing together of the endless nonwoven fabric (8) and the body-remote fiat material web, with the interposition of the elasticating means (12), and fixing of the elasticating means (12) by the first and second adhesive coating (24, 26),
deelastication of the elasticating means (12), in a region (28) which has remained free of adhesive in the course of the clocking of the second adhesive coating (26), in the product longitudinal direction (14).

23. The method as claimed in claim 22, further characterized by all-over application of a third adhesive coating (36), in the machine direction (13), to the body-remote side (22) of the nonwoven fabric (8) in the bilateral marginal regions (20) outside of the second adhesive coating (26) and outside of the bilateral elasticating means (12) in the product transverse direction (16).

24. The method as claimed in claim 23, further characterized by all-over application of a fourth adhesive coating (46) in the machine direction (13) to the body-remote side (22) of the nonwoven fabric (8) onto a region in the product transverse direction (16) between the bilateral first adhesive coating (24) in the form of adjacently extended adhesive tracks (48).

25. The method as claimed in claim 24, characterized in that the third adhesive coating (36) and/or the fourth adhesive coating (46) are applied continuously in the machine direction (13).

26. The method as claimed in claim 22, characterized in that, for the fixing of the elasticating means (12), only the first and the second adhesive coating (24, 26) are used and the body-remote flat material web remains uncoated in this region.

27. The method as claimed in claim 24, characterized in that, for the application of the first and the second adhesive coating (24, 26), in particular also for the application of the third and/or fourth adhesive coating (36, 46), an adhesive coating apparatus (42, 44), which in closest contact to the nonwoven fabric delivers adhesive material from, in particular, slit-shaped adhesive discharge openings, is used.

28. The method as claimed in claim 22, characterized in that, after the nonwoven fabric (8) and the body-remote flat material web have been brought together, contour cuts, conducted in the machine direction (13), are made on both sides in order to form the leg openings or the leg cutouts (18).

29. The method as claimed claim 22, characterized in that an endless web of layered structures (2), respectively comprising an absorption body (10), is herewith produced, which endless web comprises the body-remote flat material web and the body-facing nonwoven fabric (8) and an endless succession of absorption bodies (10), fed one after another in the machine direction (13), which are conveyed and arranged between the body-remote flat material web and the body-facing nonwoven fabric (8), and in that, transversely to the machine direction (13), a separation cut for the formation of the layered structures (2) is performed, in that the separated layered structures (2) are rotated through 90° and are brought into bridging arrangement with an abdominal portion web (60) and a spinal portion web (62) and are non-detachably fixed in the overlap region with the abdominal portion web (60) and the spinal portion web (64).

30. The sanitary article as claimed in claim 1, characterized in that the first and/or the second adhesive coating (24, 26) has a weight per unit area of no more than 20 g/m².

31. The sanitary article as claimed in claim 11, characterized in that additionally the first adhesive coating (24), through the lateral contouring of the bilateral marginal regions (20) to form leg openings or leg cutouts (18), are in some sections configured marginally flush with longitudinal margins (34) of the nonwoven fabric (8).

32. The sanitary article as claimed in claim 15, characterized in that the nonwoven fabric (8) has in particular a weight per unit area of no more than 30 g/m².

33. The sanitary article as claimed in claim 1 is a diaper, a pants-type diaper or an incontinence pad.

34. The method as claimed in claim 24, further characterized by all-over application of a fourth adhesive coating (46) in the machine direction (13) to the body-remote side (22) of the nonwoven fabric (8) onto a region in the product transverse direction (16) between the bilateral first adhesive coating (24) in the form of adjacently extended adhesive tracks (48) having a width of 1-5 mm and at a distance apart of the adhesive tracks of 2-20 mm.

35. The method as claimed in claim 22, further characterized by all-over application of a fourth adhesive coating (46) in the machine direction (13) to the body-remote side (22) of the nonwoven fabric (8) onto a region in the product transverse direction (16) between the bilateral first adhesive coating (24) in the form of adjacently extended adhesive tracks (48).
